(19) United States
(12) Patent Application Publication
(10) Pub. No.: US 2011/0212169 A1
(43) Pub. Date: Sep. 1, 2011

(54) METHOD FOR PRODUCING POWDER CONTAINING NANOPARTICULATED SPARINGLY SOLUBLE DRUG, POWDER PRODUCED THEREBY AND PHARMACEUTICAL COMPOSITION CONTAINING SAME (As Amended)

(75) Inventors: Joon-Ho Bae, Gyeonggi-do (KR); Hyeok Lee, Gyeonggi-do (KR); Deok-Ki Hong, Gyeonggi-do (KR); Jong-Hwi Lee, Seoul (KR)

(73) Assignee: Amorepacific Corporation, Yongsan-gu (KR)

(21) Appl. No.: 13/127,957

(22) PCT Filed: Nov. 10, 2009
(86) PCT No.: PCT/KR2009/006585

§ 371 Date: May 5, 2011

(30) Foreign Application Priority Data
Nov. 10, 2008 (KR) 1020080111205

(51) Int. Cl. A61K 9/14; A61K 9/48; A61K 9/20; A61K 31/496; A61K 31/439; A61K 31/216; A61K 31/192; A61P 17/00; A61P 37/06; A61P 1/00; A61P 31/10; A61P 3/06; B29B 9/12

(52) U.S. Cl. 424/451; 424/400; 424/464; 514/254.07; 514/291; 514/543; 514/568; 264/11

(57) ABSTRACT

Disclosed are a method for preparing a powder containing a nanoparticulated sparingly soluble drug, a powder prepared thereby, and a pharmaceutical composition containing the same. The disclosed method includes: providing a uniformly dispersed solution of a sparingly soluble drug which is formed into nanoparticles in the presence of a surface stabilizer; mixing the uniformly dispersed solution with a water-soluble dispersant solution; and drying the mixed solution to obtain the powder.

When the powder containing the nanoparticulated sparingly soluble drug obtained by the disclosed method is redispersed in an aqueous solution, the sparingly soluble drug

retains a particle size in the nano scale while the solubility and the dissolution rate of the drug are increased, thereby providing enhanced bioavailability. Consequently, the present disclosure can be useful in the development of preparations of a sparingly soluble drug for oral or parenteral administration.

### TECHNICAL FIELD

The present disclosure relates to a method for preparing a powder containing a nanoparticulated sparingly soluble drug, a powder prepared thereby, and a pharmaceutical composition containing the same.

### BACKGROUND ART

A sparingly soluble drug which is poorly soluble in water or a pharmaceutical composition containing the same may exhibit low bioavailability upon oral administration since it may be excreted before being absorbed in the gastrointestinal tract.

Furthermore, since it is difficult to be prepared for parenteral administration such as injection, various co-solvents or surfactants have to be used, which may cause side effects or poor patient compliance.

Although there have been attempts to prepare a nanoparticulated sparingly soluble drug in order to improve solubility in water and bioavailability of the sparingly soluble drug, it is still difficult to improve bioavailability since the nanoparticles tend to aggregate when they are redispersed in an aqueous solution.

### DISCLOSURE

### Technical Problem

The present disclosure is directed to providing a method for preparing a powder containing a sparingly soluble drug, capable of improving solubility in water and bioavailability of the sparingly soluble drug.

The present disclosure is also directed to providing a powder containing a sparingly soluble drug, which is prepared by the method.

The present disclosure is also directed to providing a pharmaceutical composition including the powder.

### Technical Solution

In one general aspect, the present disclosure provides a method for preparing a powder containing a nanoparticulated sparingly soluble drug, including: providing a uniformly dispersed solution of a sparingly soluble drug which is formed into nanoparticles in the presence of a surface stabilizer; mixing the uniformly dispersed solution with a water-soluble dispersant solution; and drying the mixed solution to obtain the powder.

The water-soluble dispersant may be at least one selected from carageenan, gelatin, agar, alginic acid, arabinoxylan gum, β-glucan, guar gum, arabia gum, locust bean gum, pectin, starch, xanthan gum, casein, glucomannan, cyclodextrin, methylcellulose, chitosan, xyloglucan and gluten. Specifically, it may be carageenan.

The water-soluble dispersant solution may have a concentration of about 0.1-5 wt %, and the water-soluble dispersant solution may be used in an amount of about 0.01-0.1 wt % based on the weight of the sparingly soluble drug.

The sparingly soluble drug may be at least one selected, for example, from: a nonsteroidal anti-inflammatory drug including acetaminophen, acetylsalicylic acid, ibuprofen, fenbuprofen, fenoprofen, flurbiprofen, indomethacin, naproxen, etodolac, ketoprofen, dexibuprofen, piroxicam or aceclofenac; an immunosuppressant or atopic dermatitis drug including cyclosporin, tacrolimus, rapamycin, mycophenolate or pimecrolimus; a calcium channel blocker including nifedipine, nimodipine, nitrendipine, nilvadipine, felodipine, amlodipine or isradipine; an angiotensin II antagonist including valsartan, eprosartan, irbesartan, candesartan, telmisartan, olmesartan or losartan; a cholesterol synthesis-inhibiting hypolipidemic agent including atorvastatin, lovastatin, simvastatin, fluvastatin, rosuvastatin or pravastatin; a cholesterol metabolism- and secretion-promoting hypolipidemic agent including gemfibrozil, fenofibrate, etofibrate or bezafibrate; an antidiabetic drug including pioglitazone, rosiglitazone or metformin; a lipase inhibitor including orlistat; an antifungal agent including itraconazole, amphotericin B, terbinafine, nystatin, griseofulvin, fluconazole or ketoconazole; a hepatoprotective drug including biphenyl dimethyl dicarboxylate, silymarin or ursodeoxycholic acid; a gastrointestinal drug including sofalcone, omeprazole, pantoprazole, famotidine, itopride or mesalazine; an antiplatelet agent including cilostazol or clopidogrel; an osteoporosis drug including raloxifene; an antiviral drug including acyclovir, famciclovir, lamivudine or oseltamivir; an antibiotic including clarithromycin, ciprofloxacin or cefuroxime; an antiasthmatic or antihistamine drug including pranlukast, budesonide or fexofenadine; a hormone drug including testosterone, prednisolone, estrogen, cortisone, hydrocortisone or dexamethasone; an anticancer drug including paclitaxel, docetaxel, paclitaxel derivatives, doxorubicin, adriamycin, daunomycin, camptothecin, etoposide, teniposide or busulfan; salts thereof; and pharmaceutical derivatives thereof. Specifically, it may be at least one selected from naproxen, tacrolimus, valsartan, simvastatin, fenofibrate, itraconazole, biphenyl dimethyl dicarboxylate, silymarin, sofalcone, pantoprazole, cilostazol, salts thereof and pharmaceutical derivatives thereof.

For example, the surface stabilizer may be at least one selected from sodium dodecyl sulfate, dioctyl sodium sulfosuccinate, lecithin, phospholipid, polyoxyethylene sorbitan fatty acid ester, potassium sorbate, poloxamer, propylene glycol, methyl cellulose, ethyl cellulose, hydroxymethyl cellulose, hydroxyethyl cellulose, hydroxypropyl cellulose, hydroxypropyl methylcellulose, carboxymethyl cellulose, benzethonium chloride, benzalconium chloride, sorbic acid, potassium sorbate, benzoic acid, sodium benzoate, propylparaben, methylparaben, polyvinyl alcohol, polyvinylpyrrolidone, alginic acid and sodium alginate. The surface stabilizer may be used in an amount of about 0.0001-90 wt % based on the weight of the sparingly soluble drug.

The uniformly dispersed solution may have an apparent viscosity ranging from 1 to 100,000 centipoises.

In another general aspect, the present disclosure provides a powder containing a nanoparticulated sparingly soluble drug, comprising: a sparingly soluble drug which is formed into nanoparticles in the presence of a surface stabilizer; and a water-soluble dispersant; when the powder is redispersed in an aqueous solution, 10 to 90% of the particles based on a particle size normal distribution curve have a particle size ranging from 10 to 1,000 nm. Specifically, the 10 to 90% of the particles may have a particle size of about 10 to 400 nm based on the particle size normal distribution curve.

In another general aspect, the present disclosure provides a pharmaceutical composition comprising the powder containing the nanoparticulated sparingly soluble drug.

A formulation of the pharmaceutical composition may be granule, powder, syrup, liquid, suspension, tablet, capsule, troche or pill for oral administration, or transdermal agent, lotion, ophthalmic ointment, ointment, plaster, cataplasm, cream, paste, suspension, liquid, injection or suppository for parenteral administration.

### Advantageous Effects

When the powder containing the nanoparticulated sparingly soluble drug obtained by the method according to the present disclosure is redispersed in an aqueous solution, the sparingly soluble drug retains a particle size in the nano scale while the solubility and the dissolution rate of the drug are increased, thereby providing enhanced bioavailability. Consequently, the present disclosure can be useful in the development of preparations of a sparingly soluble drug for oral or parenteral administration.

### DESCRIPTION OF DRAWINGS

FIG. 1 shows particle size distribution of the powders containing the drug prepared in Example 1 when redispersed in an aqueous solution, depending on the concentration of a carageenan solution;

FIGS. 2a and 2b show electron microscopic images of the powders containing the drug prepared in Example 1 when redispersed in the aqueous solution, when the concentration of the carageenan solution is 0.1 wt % (2a) and 2 wt % (2b); and

FIGS. 3a and 3b show atomic force microscopic (AFM) images of the powders containing the drug prepared in Example 5 when redispersed in an aqueous solution, when the concentration of the carageenan solution is 0.5 wt % (3a) and 1 wt % (3b).

### MODE FOR INVENTION

Hereinafter, the embodiments of the present disclosure will be described in detail.

Step 1: Provision of Uniformly Dispersed Solution Containing Sparingly Soluble Drug

In the step 1, an active ingredient, i.e. a sparingly soluble drug, is mixed with a surface stabilizer. Then, the resulting mixture is ground and dispersed uniformly.

In the present disclosure, the sparingly soluble drug used as the active ingredient is not particularly restricted, but may be an organic material which is sparingly soluble in a liquid dispersant. The liquid dispersant may be water or an aqueous solution. Alternatively, it may be an alcohol or an oil. “Sparingly soluble” as used herein means a solubility of 30 mg/mL or less, specifically, 10 mg/mL or less, more specifically, 0.1 mg/mL or less, in a liquid dispersant at room temperature.

Specific examples of the sparingly soluble drug may include: a nonsteroidal anti-inflammatory drug including acetaminophen, acetylsalicylic acid, ibuprofen, fenbuprofen, fenoprofen, flurbiprofen, indomethacin, naproxen, etodolac, ketoprofen, dexibuprofen, piroxicam or aceclofenac; an immunosuppressant or atopic dermatitis drug including cyclosporin, tacrolimus, rapamycin, mycophenolate or pimecrolimus; a calcium channel blocker including nifedipine, nimodipine, nitrendipine, nilvadipine, felodipine, amlodipine or isradipine; an angiotensin II antagonist including valsartan, eprosartan, irbesartan, candesartan, telmisartan, olmesartan or losartan; a cholesterol synthesis-inhibiting hypolipidemic agent including atorvastatin, lovastatin, simvastatin, fluvastatin, rosuvastatin or pravastatin; a cholesterol metabolism- and secretion-promoting hypolipidemic agent including gemfibrozil, fenofibrate, etofibrate or bezafibrate; an antidiabetic drug including pioglitazone, rosiglitazone or metformin; a lipase inhibitor including orlistat; an antifungal agent including itraconazole, amphotericin B, terbinafine, nystatin, griseofulvin, fluconazole or ketoconazole; a hepatoprotective drug including biphenyl dimethyl dicarboxylate, silymarin or ursodeoxycholic acid; a gastrointestinal drug including sofalcone, omeprazole, pantoprazole, famotidine, itopride or mesalazine; an antiplatelet agent including cilostazol or clopidogrel; an osteoporosis drug including raloxifene; an antiviral drug including acyclovir, famciclovir, lamivudine or oseltamivir; an antibiotic including clarithromycin, ciprofloxacin or cefuroxime; an antiasthmatic or antihistamine drug including pranlukast, budesonide or fexofenadine; a hormone drug including testosterone, prednisolone, estrogen, cortisone, hydrocortisone or dexamethasone; an anticancer drug including paclitaxel, docetaxel, paclitaxel derivatives, doxorubicin, adriamycin, daunomycin, camptothecin, etoposide, teniposide or busulfan; therapeutically equivalent salts thereof; and pharmaceutical derivatives thereof.

Specifically, the sparingly soluble drug may be at least one selected from naproxen, tacrolimus, valsartan, simvastatin, fenofibrate, itraconazole, biphenyl dimethyl dicarboxylate, silymarin, sofalcone, pantoprazole, cilostazol, salts thereof, and pharmaceutical derivatives thereof.

The particle size of the sparingly soluble drug used in the step 1 is not particularly restricted. For example, the sparingly soluble drug may be pretreated using a commonly employed milling method such as fragmentation or air jet milling to form particles having an average particle size of less than 100 μm, before conducting the step 1.

The surface stabilizer serves to prevent aggregation of the sparingly soluble drug particles. It can be any of pharmaceutically acceptable organic or inorganic compounds which are physically miscible with the sparingly soluble drug and the water-soluble dispersant but do not chemically react them.

Representative examples may include sodium dodecyl sulfate (SDS or SLS), dioctyl sodium sulfosuccinate, lecithin, phospholipid, polyoxyethylene sorbitan fatty acid ester (e.g., Tween), potassium sorbate, poloxamer, propylene glycol, methyl cellulose, ethyl cellulose, hydroxymethyl cellulose, hydroxyethyl cellulose, hydroxypropyl cellulose, hydroxypropyl methylcellulose, carboxymethyl cellulose, benzethonium chloride, benzalconium chloride, sorbic acid, potassium sorbate, benzoic acid, sodium benzoate, propylparaben, methylparaben, polyvinyl alcohol, polyvinylpyrrolidone, alginic acid, sodium alginate, and a mixture thereof. Specifically, it may be at least one selected from hydroxypropyl cellulose and poloxamer.

In the present disclosure, the surface stabilizer may be used in an amount of 0.0001-90 wt %, specifically 0.01-50 wt %, more specifically 0.1-20 wt %, based on the weight of the sparingly soluble drug.

When grinding the mixture of the sparingly soluble drug and the surface stabilizer, water, an aqueous solution or a buffer solution may be used as a solvent. The solvent may contain an alcohol in an amount of less than 50% depending on the properties of the sparingly soluble drug. The alcohol that may be employed in the present disclosure includes methyl alcohol, ethyl alcohol, propyl alcohol, etc., and a mixture thereof.

In the present disclosure, an aqueous solution containing the sparingly soluble drug and the surface stabilizer is mixed and ground using mechanical energy to reduce the particle size of the sparingly soluble drug and homogenize the dispersion.

The grinding may be conducted by a commonly employed method, for example, by a wet grinding process using a dispersion mill such as a ball mill, an oscillating mill, a bead mill, etc., an ultrasonic irradiation process, a shearing force grinding process, or the like. The processing temperature and processing time may be adjusted appropriately according to the kind of the sparingly soluble drug and mechanical properties thereof. For example, the grinding may be conducted at room temperature, and the grinding time may be varied according to mechanical means and processing conditions. For example, ball milling may be conducted for 3 days or longer when a low shear energy is used, and it may be finished in several hours when a high shear energy is employed.

The sparingly soluble drug may be formed into nanoparticles by the grinding. That is to say, the sparingly soluble drug may be ground such that it exhibits a particle size distribution of 10 to 1,000 nm, specifically 10 to 400 nm, for 10 to 90% of the drug particles determined based on a particle size normal distribution curve.

The uniformly dispersed solution obtained in the step 1 has an apparent viscosity ranging from 1 to 100,000 centipoises, specifically 10-50,000 centipoises, more specifically 500-10,000 centipoises. As the processing time of the step 1 is longer, the particle size of the sparingly soluble drug becomes smaller and more uniform.

Step 2: Mixing of Uniformly Dispersed Solution Containing Drug with Water-Soluble Dispersant Solution

In the step 2 of the present disclosure, the uniformly dispersed solution containing drug obtained in the step 1 is mixed with the water-soluble dispersant solution for aiding in dispersion by stirring for several minutes to several hours so as to prevent aggregation of the drug during drying, maintain the particle size of the drug in the nano scale even in the powder state, and retain the particle size in the nano scale even when redispersed in an aqueous solution.

The water-soluble dispersant used in the present disclosure may be a polymer material that dissolves well and is viscous in water, and is unharmful to the human body. Representative examples may include polysaccharides such as carageenan, gelatin, agar, alginic acid, arabinoxylan gum, β-glucan, guar gum, arabia gum, locust bean gum, pectin, starch, xanthan gum, casein, glucomannan, cyclodextrin, methylcellulose, chitosan, xyloglucan and gluten, etc. These may be used alone or in combination. Specifically, carageenan, gelatin or alginic acid, etc. may be used among them. Most specifically, carageenan may be used. The water-soluble dispersant solution may have a concentration of 0.1-5 wt %, specifically 2-5 wt %.

The water-soluble dispersant solution may be used in an amount of 0.01-0.1 wt % based on the weight of the sparingly soluble drug. Since the water-soluble dispersant solution is capable of preventing aggregation of the sparingly soluble drug and maintaining the particle size of the drug in the nano scale even with a small amount, the powder containing the sparingly soluble drug according to the present disclosure may include significantly decreased amount of an excipient and thus may improve patient compliance.

Step 3: Obtainment of Powder

In the step 3, the mixed dispersion solution obtained in the step 2 is dried by a commonly employed process to obtain powder.

By evaporating water from the mixed dispersion solution through freeze drying, vacuum drying or hot air drying, the powder of the present disclosure may be obtained.

The powder obtained in accordance with the present disclosure retains the original particle size in the nano scale when the powder is redispersed in an aqueous solution such as water or a buffer solution, and 10 to 90% of the particles based on a particle size normal distribution curve have a particle size of 10 to 1,000 nm, specifically 10 to 400 nm.

Since the powder prepared according to the present disclosure, in which the sparingly soluble drug, the surface stabilizer and the water-soluble dispersant solution are uniformly mixed, stably retains the original particle size in the nano scale when redispersed in water or an aqueous solution, it exhibits enhanced bioavailability without side effects caused by impurities. The powder prepared according to the present disclosure may retain the particle size in the nano scale at room temperature for 6 months or longer when redispersed in an aqueous solution, without aggregation.

Furthermore, it can be stored easily since it is in powder form, and it may be useful in the development of preparations for oral or parenteral administration.

The present disclosure further provides a pharmaceutical composition comprising the powder prepared according to the present disclosure together with a commonly employed pharmaceutically acceptable carrier. A formulation of the pharmaceutical composition may be granule, powder, syrup, liquid, suspension, tablet, capsule, troche or pill for oral administration, or transdermal agent, lotion, ophthalmic ointment, ointment, plaster, cataplasm, cream, paste, suspension, liquid, injection or suppository for parenteral administration.

The examples and experiments will now be described. The following examples and experiments are for illustrative purposes only and not intended to limit the scope of the present disclosure.

### Example 1

### Particle Size Variation of Drug Depending on Concentration of Water-Soluble Dispersant Solution In order to observe particle size variation of a drug depending on the concentration of a water-soluble dispersant solution, naproxen (TCI Chem) was used as a sparingly soluble drug and a carageenan solution was used as the water-soluble dispersant solution.

Specifically, hydroxypropyl cellulose (hereinafter HPC, 0.33 g) and distilled water (22.67 g) were added to naproxen (2 g), and the mixture was wet ground at room temperature for 5 days using Micro Jet Mill System (JE Powder, Korea). The resulting slurry mixture was mixed with a carageenan solution of the same amount. At this time, the concentration of the carageenan solution was varied at 5, 3, 2, 1, 0.5, 0.1 and 0 wt %. When the concentration was 0 wt %, distilled water was added instead of the carageenan solution. The resulting mixture was frozen in a refrigerator and freeze dried for 24 hours using a freeze drier to obtain the desired powder.

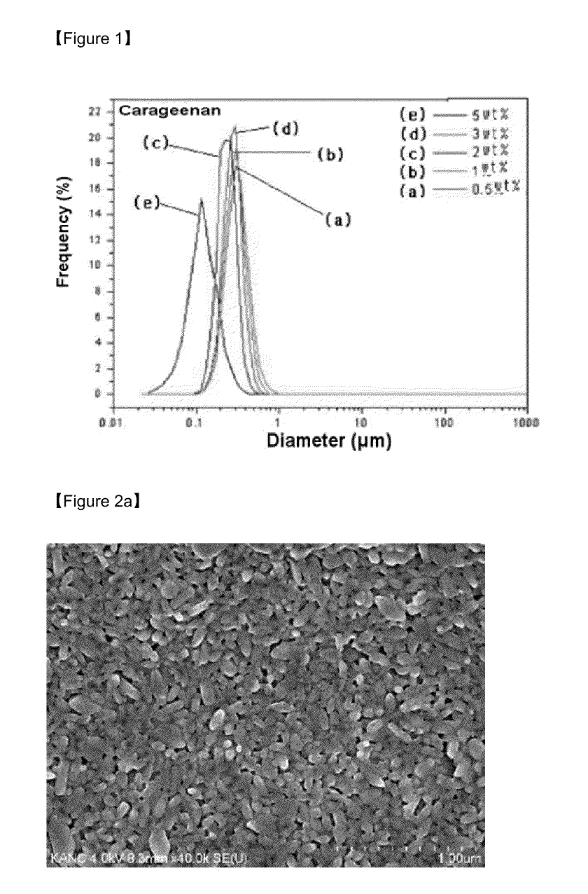

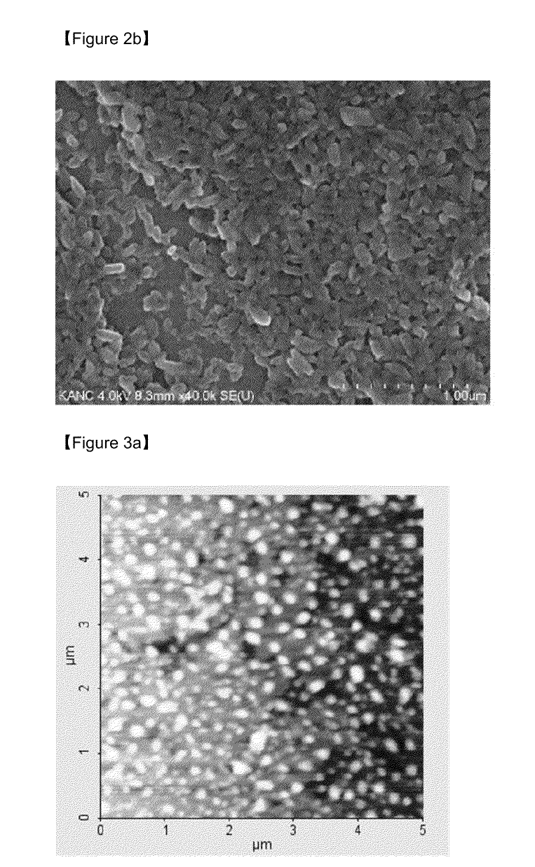

Each powder (0.01 g) was redispersed in distilled water (5 mL) and particle size was measured. The result is shown in FIG. 1. Electron microscopic images of the powders when the concentration of the carageenan solution was 0.1 wt % and 2 wt % are shown in FIGS. 2a and 2b, respectively. The redispersion was conducted by lightly shaking the mixture with a hand. The particle size was measured under aqueous condition using a laser scattering particle size analyzer (LA 910, Horiba, Japan) (Mie & Fraunhofer, relative refraction index=1). The resolving power of the ultrasonic disperser used for the particle size measurement was 40 W (39 kHz), and the speed of stirring and circulation was 340 mL/min. The particle size measurement was made after performing ultrasonic dispersion for 1 minute.

When the slurry mixture obtained after the wet grinding was redispersed in distilled water, the average particle size of the naproxen particles was 0.10 μm, and when the dried powder was redispersed in distilled water, the average particle size of the naproxen particles depending on the concentration of carageenan was as follows.

TABLE 1

| Carageenan | Average particle |
|---|---|
| concentration (wt %) | size (μm) |
| 5 | 0.12 (±0.05) |
| 3 | 0.26 (±0.07) |
| 2 | 0.23 (±0.06) |
| 1 | 0.28 (±0.08) |
| 0.5 | 0.31 (±0.10) |
| 0.1 | 0.91 (±2.00) |
| 0 (Comparative | 16.2 (±14.4) |
| Example 1) | |

As seen from Table 1, when the carageenan concentration was 0 wt % (Comparative Example 1), the drug particle size was as large as 16.2 μm. In contrast, even when the carageenan concentration was as low as 0.1 wt %, the nano-scale particle size was retained. As the carageenan concentration was increased, the drug particle size decreased. Also, as seen from FIGS. 1, 2a and 2b, the drug particle size was retained in the nano scale when the carageenan concentration was from 0.5 to 5 wt %.

### Example 2

### Particle Size Variation of Drug Depending on Drying Method

<Vacuum Drying at Room Temperature>

In order to observe particle size variation of a drug depending on the drying method, powders were prepared in the same manner as in Example 1, except for changing the carageenan concentration to 3, 2, 1 and 0.5 wt % and vacuum drying at room temperature.

The obtained powders were redispersed in distilled water and drug particle size was measured in the same manner as in Example 1. The result is given in Table 2.

TABLE 2

| Carageenan | Average particle |
|---|---|
| concentration (wt %) | size (μm) |
| 3 | 0.23 (±0.06) |
| 2 | 0.21 (±0.06) |
| 1 | 0.26 (±0.06) |
| 0.5 | 0.36 (±0.71) |
| 0 (Comparative | 22.2 (±16.1) |
| Example 2) | |

As seen from Table 2, when the carageenan concentration was 0 wt % (Comparative Example 2), the drug particle size increased as compared to when freeze drying was performed (Comparative Example 1). In contrast, when carageenan was used, the nano-scale particle size was retained after the redispersion.

<Hot Air Drying>

Powders were prepared in the same manner as in Example 1, except for using a 1 wt % carageenan solution and hot air drying at 60° C. and 40° C.

The obtained powders were redispersed in distilled water and drug particle size was measured in the same manner as in Example 1. The result is given in Table 3.

TABLE 3

| Hot air drying | Average particle |
|---|---|
| temperature (° C.) | size (μm) |
| 60 | 0.36 (±0.29) |
| 40 | 0.39 (±1.01) |

As seen from Table 3, the nano-scale particle size was retained after the redispersion even when hot air drying was performed at 40° C. and 60° C.

### Example 3

### Particle Size Variation of Drug Depending on Kind of Water-Soluble Dispersant Solution and Drying Method

Powders were prepared in the same manner as in Example 1, except for using gelatin or alginic acid solution instead of the carageenan solution and performing vacuum drying at room temperature or freeze drying.

The obtained powders were redispersed in distilled water and drug particle size was measured in the same manner as in Example 1. The result is given in Tables 4 and 5.

TABLE 4

| | |
|---|---|
| Dispersant | Vacuum drying at room temperature |

| concentration (wt %) | Gelatin | Alginic acid |
|---|---|---|
| 3 | 0.36 (±0.10) | — |
| 2 | — | 0.19 (±0.05) |
| 1 | — | 0.31 (±0.41) |

TABLE 5

| Dispersant | Freeze drying |
|---|---|
| concentration (wt %) | Gelatin |
| 5 | 0.16 (±0.05) |
| 3 | 0.13 (±0.06) |
| 2 | 0.16 (±0.08) |
| 1 | 0.45 (±0.89) |

As seen from Tables 4 and 5, when the gelatin or alginic acid solution was used as the water-soluble dispersant solution, the nano-scale particle size was retained after the redispersion for the different drying methods.

### Example 4

### Particle Size Variation of Drug Depending on Drug Concentration

Powders were prepared in the same manner as in Example 1, except for increasing the concentration of naproxen to 16 wt % and changing the carageenan concentration to 1 and 0.5 wt %.

The obtained powders were redispersed in distilled water and drug particle size was measured in the same manner as in Example 1. The result is given in Table 6.

TABLE 6

| Carageenan | Average particle |
|---|---|
| concentration (wt %) | size (μm) |
| 1 | 0.22 (±0.07) |
| 0.5 | 0.56 (±1.20) |

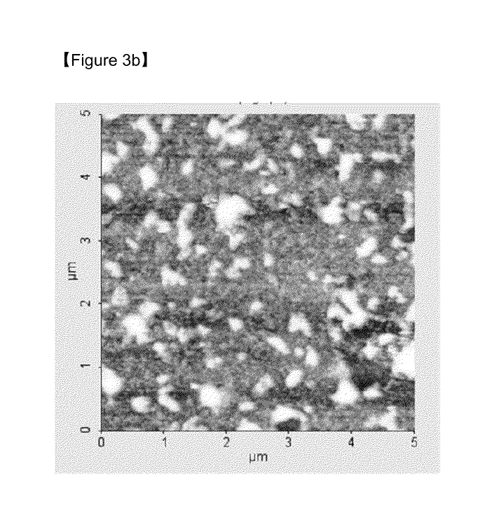

As seen from Table 6, the nano-scale particle size was retained after the redispersion even when the drug concentration was increased. Furthermore, the redispersed dried nanoparticle powders were observed by atomic force microscopy (AFM). As seen from FIGS. 3a and 3b, the nano-scale particle size was retained after the redispersion.

### Example 5

### Particle Size Variation of Drug Depending on Kind of Drug

Powders were prepared in the same manner as in Example 1, except for using itraconazole (Pacific Pharma), tacrolimus (Pacific Pharma), fenofibrate (Sigma) and sofalcone (Dae Hee Chemical) and changing the carageenan concentration to 5 wt %.

The obtained powders were redispersed in distilled water and drug particle size was measured in the same manner as in Example 1. The result is given in Table 7.

TABLE 7

| | Average particle |
|---|---|
| Drugs | size (μm) |
| Itraconazole | 0.11 (±0.06) |
| Tacrolimus | 0.16 (±0.09) |
| Fenofibrate | 0.16 (±0.09) |
| Sofalcone | 0.32 (±0.11) |

As seen from Table 7, the nano-scale particle size was retained after the redispersion for the different drugs when the carageenan solution was used as the water-soluble dispersant solution.

1. A method for preparing a powder containing a nanoparticulated sparingly soluble drug, comprising:
providing a uniformly dispersed solution of a sparingly soluble drug which is formed into nanoparticles in the presence of a surface stabilizer;
mixing the uniformly dispersed solution with a water-soluble dispersant solution; and
drying the mixed solution to obtain the powder.

2. The method according to claim 1, wherein the water-soluble dispersant is at least one selected from carageenan, gelatin, agar, alginic acid, arabinoxylan gum, β-glucan, guar gum, arabia gum, locust bean gum, pectin, starch, xanthan gum, casein, glucomannan, cyclodextrin, methylcellulose, chitosan, xyloglucan and gluten.

3. The method according to claim 2, wherein the water-soluble dispersant is carageenan.

4. The method according to claim 1, wherein the water-soluble dispersant solution has a concentration of 0.1-5 wt %.

5. The method according to claim 1, wherein the water-soluble dispersant solution is used in an amount of 0.01-0.1 wt % based on the weight of the sparingly soluble drug.

6. The method according to claim 1, wherein the sparingly soluble drug is at least one selected from: a nonsteroidal anti-inflammatory drug including acetaminophen, acetylsalicylic acid, ibuprofen, fenbuprofen, flurbiprofen, indomethacin, naproxen, etodolac, ketoprofen, dexibuprofen, piroxicam or aceclofenac; an immunosuppressant or atopic dermatitis drug including cyclosporin, tacrolimus, rapamycin, mycophenolate or pimecrolimus; a calcium channel blocker including nifedipine, nimodipine, nitrendipine, nilvadipine, felodipine, amlodipine or isradipine; an angiotensin II antagonist including valsartan, eprosartan, irbesartan, candesartan, telmisartan, olmesartan or losartan; a cholesterol synthesis-inhibiting hypolipidemic agent including atorvastatin, lovastatin, simvastatin, fluvastatin, rosuvastatin or pravastatin; a cholesterol metabolism- and secretion-promoting hypolipidemic agent including gemfibrozil, fenofibrate, etofibrate or bezafibrate; an antidiabetic drug including pioglitazone, rosiglitazone or metformin; a lipase inhibitor including orlistat; an antifungal agent including itraconazole, amphotericin B, terbinafine, nystatin, griseofulvin, fluconazole or ketoconazole; a hepatoprotective drug including biphenyl dimethyl dicarboxylate, silymarin or ursodeoxycholic acid; a gastrointestinal drug including sofalcone, omeprazole, pantoprazole, famotidine, itopride or mesalazine; an antiplatelet agent including cilostazol or clopidogrel; an osteoporosis drug including raloxifene; an antiviral drug including acyclovir, famciclovir, lamivudine or oseltamivir; an antibiotic including clarithromycin, ciprofloxacin or cefuroxime; an antiasthmatic or antihistamine agent including pranlukast, budesonide or fexofenadine; a hormone drug including testosterone, prednisolone, estrogen, cortisone, hydrocortisone or dexamethasone; an anticancer drug including paclitaxel, docetaxel, paclitaxel derivatives, doxorubicin, adriamycin, daunomycin, camptothecin, etoposide, teniposide or busulfan; salts thereof; and pharmaceutical derivatives thereof.

7. The method according to claim 6, wherein the sparingly soluble drug is at least one selected from naproxen, tacrolimus, valsartan, simvastatin, fenofibrate, itraconazole, biphenyl dimethyl dicarboxylate, silymarin, sofalcone, pantoprazole, cilostazol, salts thereof and pharmaceutical derivatives thereof.

8. The method according to claim 1, wherein the surface stabilizer is at least one selected from sodium dodecyl sulfate, dioctyl sodium sulfosuccinate, lecithin, phospholipid, polyoxyethylene sorbitan fatty acid ester, potassium sorbate, poloxamer, propylene glycol, methyl cellulose, ethyl cellulose, hydroxymethyl cellulose, hydroxyethyl cellulose, hydroxypropyl cellulose, hydroxypropyl methylcellulose, carboxymethyl cellulose, benzethonium chloride, benzalconium chloride, sorbic acid, potassium sorbate, benzoic acid, sodium benzoate, propylparaben, methylparaben, polyvinyl alcohol, polyvinylpyrrolidone, alginic acid, and sodium alginate.

9. The method according to claim 1, wherein the surface stabilizer is used in an amount of 0.0001-90 wt % based on the weight of the sparingly soluble drug.

10. The method according to claim 1, wherein the uniformly dispersed solution has an apparent viscosity ranging from 1 to 100,000 centipoises.

11. A powder containing a nanoparticulated sparingly soluble drug, comprising:
a sparingly soluble drug which is formed into nanoparticles in the presence of a surface stabilizer; and
a water-soluble dispersant,
wherein 10 to 90% of the particles based on a particle size normal distribution curve have a particle size ranging from 10 to 1,000 nm when the powder is redispersed in an aqueous solution.

12. The powder according to claim 11, wherein 10 to 90% of the particles based on the particle size normal distribution curve have a particle size ranging from 10 to 400 nm when the powder is redispersed in the aqueous solution.

13. A pharmaceutical composition comprising the powder of claim 11 together with a pharmaceutically acceptable carrier.

14. The pharmaceutical composition according to claim 13, wherein the formulation of pharmaceutical composition is granule, powder, syrup, liquid, suspension, tablet, capsule, troche or pill for oral administration; or transdermal agent, lotion, ophthalmic ointment, ointment, plaster, cataplasm, cream, paste, suspension, liquid, injection or suppository for parenteral administration.
